(19) United States
(12) Patent Application Publication
(10) Pub. No.: US 2010/0137725 A1
(43) Pub. Date: Jun. 3, 2010

(54) SPHYGMOMANOMETER CUFF AND SPHYGMOMANOMETER

(76) Inventors: Akihisa Takahashi, Kyoto (JP); Yoshihide Onishi, Kyoto (JP); Tomohiro Kukita, Kyoto (JP); Chisato Uesaka, Kyoto (JP); Yukiya Sawanoi, Nara (JP); Masataka Yanagase, Osaka (JP)

Correspondence Address: MORRISON & FOERSTER LLP, 1650 TYSONS BOULEVARD, SUITE 400, MCLEAN, VA, 22102, US

(21) Appl. No.: 12/444,353

(22) PCT Filed: Sep. 28, 2007
(86) PCT No.: PCT/JP2007/068975

§ 371 Date: Jan. 20, 2010

(30) Foreign Application Priority Data
Oct. 5, 2006 (JP) 2006-274223

(51) Int. Cl. A61B 5/0225

(52) U.S. Cl. 600/493

(57) ABSTRACT

A sphygmomanometer cuff is used by being wrapped around an upper arm, and includes an air bladder, a cover body, and a cushion material. The air bladder is inflated/contracted by in/out of fluid, and includes a compression acting surface positioned on the upper arm side when the sphygmomanometer cuff is wrapped around the upper arm. The cover body internally includes the air bladder, and includes an inner peripheral side sheet portion positioned on the upper arm side when the sphygmomanometer cuff is wrapped around the upper arm. The cushion material is positioned on the inner peripheral side sheet portion side than the compression acting surface, and is compressible in a direction parallel to a thickness direction of the inner peripheral side sheet portion. According to such a configuration, the internal bleeding at the measuring site that may occur when measuring the blood pressure is reliably prevented.

### CROSS REFERENCE TO RELATED APPLICATIONS

This application is a National Stage application of PCT/JP2007/068975, filed Sep. 28, 2007, which claims the benefit of priority of Japanese Application No. 2006-274223, filed Oct. 5, 2006, the entire contents of these applications hereby incorporated by reference.

### TECHNICAL FIELD

The present invention relates to a sphygmomanometer cuff used by being wrapped around a measuring site of a living body such as a wrist or an upper arm when measuring blood pressure, and a sphygmomanometer equipped with the same.

### BACKGROUND ART

Normally, when measuring a blood pressure value, a cuff internally including a fluid bag for putting pressure on an artery in a living body is wrapped around the body surface of the living body, and the wrapped fluid bag is inflated and contracted to detect the arterial pressure pulse wave generated in the artery to thereby measure the blood pressure value. Here, the cuff refers to a band-shaped structural body having a lumen that can be wrapped around on one part of the living body, and refers to that which can be used to measure arterial pressure of upper and lower limbs by injecting fluid such as gas or liquid into the lumen. Therefore, the cuff is a term that represents a concept including the fluid bag and the wrapping member for wrapping the fluid bag around the living body, and in particular, the cuff worn by being wrapped around the arm or the wrist is also referred to as an arm band or a manchette.

The fluid bag in which at least two or more sheet-like members made of resin are overlapped, and the peripheral edges are joined to be formed into a bag-form is normally used. A cover body made from a fabric including a fixing portion such as a surface fastener is used for the wrapping member. The above-described fluid bag is accommodated inside the cover body, and the fluid bag and the cover body configure a sphygmomanometer cuff. With the sphygmomanometer cuff configured in this manner, the main surface on the inner peripheral side of the fluid bag positioned on the living body side when the sphygmomanometer cuff is wrapped around the living body functions as a compression acting surface for putting pressure on the living body.

The sphygmomanometer cuff described above has a problem that wrinkles form at the compression acting surface of the fluid bag when measuring the blood pressure. If wrinkles are formed at the compression acting surface of the fluid bag, part of the measuring site may be trapped in the valley portions of the wrinkles, which may cause slight internal bleeding at the measuring site. This point will be described in detail below with reference to the drawings.

FIG. 29 is a view showing a state in which a conventional sphygmomanometer cuff is wrapped around an upper arm, which is a measuring site. As shown in FIG. 29, a sphygmomanometer cuff 150X is wrapped around an upper arm 200 to measure blood pressure, and an air bladder 151, which is a fluid bag, is inflated while maintaining such a state, so that an inner sheet 152 of the air bladder 151 moves towards the upper arm 200 side with the inflation of the air bladder 151, and a compression acting surface 152a, which is a main surface on the upper arm 200 side, closely attaches to an inner peripheral side sheet portion 162 of a cover body 161. The inner peripheral side sheet portion 162 of the cover body 161 then also moves towards the upper arm 200 side, and closely attaches to the upper arm 200. In this case, the diameter of the inner sheet 152 itself of the air bladder 151 reduces, whereby an extra portion produces at the inner sheet 152, which extra portion has nowhere to escape and moves to the outer side thereby forming wrinkles S at the compression acting surface 152a. The wrinkles S are mainly formed in a direction parallel to the stretching direction of the upper arm 200.

A cross-sectional view enlarging the portion (region XXX shown in FIG. 29) where the wrinkle S is formed is shown in FIG. 30. The inner peripheral side sheet portion 162 of the cover body 161 closely attaches to the compression acting surface 152a of the air bladder 151 in the process of inflating the air bladder 151, and thus the friction between them causes the inner peripheral side sheet portion 162 of the cover body 161 to be pulled following the portion where the wrinkle S is formed of the inner sheet 151 of the air bladder 151. Thus, one part of the inner peripheral side sheet portion 162 of the cover body 161 enters the valley portions of the wrinkles S formed at the compression acting surface 152a of the air bladder 151, whereby wrinkles also form on the inner peripheral side sheet portion 162. Even if the frictional force is weak and the inner peripheral side sheet portion 162 of the cover body 161 does not follow the portion where the wrinkle S is formed of the inner sheet 152 of the air bladder 151, the diameter of the inner peripheral side sheet portion 162 of the cover body 161 also reduces with the inflation of the air bladder 151, and thus an extra portion produces at the inner peripheral side sheet portion 162 of the cover body 161. Thus, the extra portion enters the valley portions of the wrinkles S formed at the compression acting surface 152a of the air bladder 151, and wrinkles also form at the inner peripheral side sheet portion 162.

In the process of inflating the air bladder 151, the inner peripheral side sheet portion 162 closely attaches to the upper arm 200, as described above, and thus the friction between them causes one part of the skin of the upper arm 200 to be pulled following the portion where the wrinkle is formed of the inner peripheral side sheet portion 162. Thus, part of the skin of the upper arm 200 enters the valley portions of the wrinkles and is caught in the wrinkles. As a result, slight internal bleeding may occur at the relevant portion when measuring the blood pressure.

The wrinkles formed at the compression acting surface of the air bladder thus become the cause of slight internal bleeding at the measuring site of a subject. Therefore, a technique of preventing the wrinkle itself from forming at the compression acting surface of the air bladder when measuring the blood pressure, or a technique of preventing part of the skin from getting caught at the valley portion of the wrinkle even if wrinkles are formed at the compression acting surface of the air bladder is being conventionally reviewed. Representative examples are a technique disclosed in Japanese Unexamined Patent Publication No. 2000-51158 (Patent Document 1), and techniques disclosed in Japanese Unexamined Patent Publication Nos. 2006-81668 (Patent Document 2) and 2006-218178 (Patent Document 3).

The technique disclosed in Patent Document 1 is a technique in which the inner peripheral side sheet portion of the cover body has a two-layer structure, where such two sheets are formed from two fabrics that easily slide with respect to each other so that the sheet positioned on the living body side of the two sheets does not move following the sheet positioned on the air bladder side, whereby part of the skin will not be caught at the valley portions of the wrinkles even if wrinkles are formed at the compression acting surface of the air bladder.

The technique disclosed in Patent Document 2 is a technique in which a sponge member is arranged in the interior of the air bladder so that wrinkles contact the sponge member thereby preventing the wrinkles from growing any larger even if wrinkles are formed at the inner peripheral side sheet portion, whereby the wrinkles formed at the compression acting surface of the air bladder when measuring the blood pressure are shallowly dispersed.

The technique disclosed in Patent Document 3 is a technique in which the sheet-like member made of resin configuring the air bladder is formed to a thickness of smaller than or equal to 0.15 mm, so that the difference in peripheral length between the outer sheet and the inner sheet of the air bladder that occurs when the cuff is wrapped around the measuring site is reduced, whereby the wrinkles themselves are prevented from forming at the compression acting surface of the air bladder when measuring the blood pressure.

[Patent Document 1] Japanese Unexamined Patent Publication No. 2000-51158

[Patent Document 2] Japanese Unexamined Patent Publication No. 2006-81668

[Patent Document 3] Japanese Unexamined Patent Publication No. 2006-218178

### DISCLOSURE OF THE INVENTION

### Problems to be Solved by the Invention

However, when the technique disclosed in Patent Document 1 is adopted, if large wrinkles are formed at the compression acting surface of the air bladder, an extra portion produces in both sheets irrespective of the magnitude of the frictional force generated between the two sheets of the cover body, and such an extra portion enters the valley portions of the wrinkles formed at the compression acting surface of the air bladder thereby forming wrinkles at the inner peripheral side sheet portion of the cover body. Thus, part of the skin also enters the valley portions of the wrinkles, which may cause a slight internal bleeding. It is also conceivable that a frictional coefficient between the two sheets increases with repeated use or degradation over time, or by temperature and humidity environment, whereby the frictional force that is generated increases, the sliding of the two sheets degrades, and the effect of preventing the internal bleeding lowers.

Moreover, when the techniques disclosed in patent documents 2 and 3 are adopted, the size of the wrinkle that is formed may be suppressed relatively small, but it is difficult to completely vanish the wrinkles, and thus the possibility a slight internal bleeding may occur still exists.

Therefore, the present invention has been made to solve the above-described problems, and aims to reliably prevent the internal bleeding at the measuring site that may occur when measuring the blood pressure.

### Means for Solving the Problems

A sphygmomanometer cuff based on the present invention is used by being wrapped around a living body, and includes a fluid bag, a cover body, and a cushion material. The fluid bag inflates/contracts by in/out of fluid, and includes a compression acting surface positioned on the living body side when the sphygmomanometer cuff is wrapped around the living body. The cover body internally includes the fluid bag, and has an inner peripheral side sheet portion positioned on the living body side when the sphygmomanometer cuff is wrapped around the living body. The cushion material is positioned on the inner peripheral side sheet portion side than the compression acting surface, and is compressible in a direction parallel to a thickness direction of the inner peripheral side sheet portion.

According to such a configuration, the cushion material mainly enters the valley portions of the wrinkles when wrinkles are formed at the compression acting surface of the fluid bag when measuring the blood pressure, and thus there is no room for the skin to be caught at the valley portions of the wrinkles. Therefore, a sphygmomanometer cuff in which the possibility of causing internal bleeding is greatly reduced is obtained.

As a secondary effect, the fluid bag is compressed from the outside by the restoring force of the cushion material in exhausting fluid from the fluid bag during the blood pressure measurement or after the blood pressure measurement, and thus the fluid in the fluid bag can be rapidly pushed out, and an effect of more rapid blood pressure measurement can be expected. Moreover, since the cushion material is arranged between the measuring site and the fluid bag when the sphygmomanometer cuff is worn, a feeling of pressure when compressing the measuring site by the fluid bag becomes smooth, and the subject will not feel pain caused by the sudden compression. In addition, the fluid bag is protected since the compression acting surface side of the fluid bag is covered by the cushion material.

In the sphygmomanometer cuff based on the present invention, the cushion material preferably has compressibility higher than compressibility in the thickness direction of the inner peripheral side sheet portion in the direction parallel to the thickness direction of the inner peripheral side sheet portion.

A configuration that allows the cushion material to reliably enter the valley portions of the wrinkles when measuring the blood pressure is obtained by using a cushion material having compressibility higher than the compressibility in the thickness direction of the inner peripheral side sheet portion of the cover body. Therefore, a sphygmomanometer cuff in which the possibility of causing internal bleeding is more reliably reduced is obtained.

In the sphygmomanometer cuff based on the present invention, the cushion material is preferably an independently foamed or simultaneously foamed sponge member made of rubber or synthetic resin.

The internal bleeding can be more reliably prevented by using an independently foamed or simultaneously foamed sponge member made of rubber or synthetic resin for the cushion material.

In the sphygmomanometer cuff based on the present invention, the cushion material may be positioned between the compression acting surface and the inner peripheral side sheet portion, or may be positioned on a side opposite to the compression acting surface side of the inner peripheral side sheet portion. In the sphygmomanometer cuff based on the present invention, the cushion material may be positioned in a space of the inner peripheral side sheet portion having a two-layer structure with the space inside.

According to such a configuration, the cushion material is positioned between the fluid bag and the measuring site when the sphygmomanometer cuff is worn in either case, and thus a sphygmomanometer cuff in which the possibility of causing internal bleeding is greatly reduced is obtained.

In the sphygmomanometer cuff based on the present invention, the cushion material may be attached to the inner peripheral side sheet portion, in which case, the attachment of the cushion material to the inner peripheral side sheet portion is preferably carried out by one of suturing, adhering, or welding, or a combination thereof. In the sphygmomanometer cuff based on the present invention, the cushion material may be attached to the compression acting surface, in which case, the attachment of the cushion material to the compression acting surface is preferably carried out by either adhering or welding, or a combination thereof.

According to such a configuration, the cushion material does not shift position, and thus a sphygmomanometer cuff in which the possibility of causing internal bleeding is more reliably reduced is obtained.

In the sphygmomanometer cuff based on the present invention, the cushion material may be a sheet-like member of even thickness, or a member in which thickness is changed in at least one of either axial direction or peripheral direction of the sphygmomanometer cuff. In the sphygmomanometer cuff based on the present invention, the cushion material may be positioned so as to cover the entire surface of the compression acting surface, or may include a plurality of divided bodies each being divided and arranged so as to face part of the compression acting surface.

Therefore, the shape of the cushion material is not particularly limited, and changes may be appropriately made.

A sphygmomanometer based on the present invention includes one of sphygmomanometer cuff described above; an inflation/contraction mechanism for inflating/contracting the fluid bag; a pressure detection unit for detecting pressure in the fluid bag; and a blood pressure value calculation unit for calculating a blood pressure value based on pressure information detected by the pressure detection unit.

According to such a configuration, a sphygmomanometer that does not cause internal bleeding at the measuring site in time of measurement is obtained.

### EFFECT OF THE INVENTION

According to the present invention, the internal bleeding that may occur when measuring the blood pressure is reliably prevented.

### BRIEF DESCRIPTION OF THE DRAWINGS

FIG. 1 is a perspective view showing an outer appearance of a sphygmomanometer according to a first embodiment of the present invention.

FIG. 2 is a function block diagram showing a configuration of the sphygmomanometer according to the first embodiment of the present invention.

FIG. 3 is a flowchart showing a flow of a blood pressure measurement process of the sphygmomanometer according to the first embodiment of the present invention.

FIG. 4 is a view showing a state in which the sphygmomanometer cuff according to the first embodiment of the present invention is developed.

FIG. 5 is a cross-sectional view for describing an internal structure of the sphygmomanometer cuff according to the first embodiment of the present invention.

FIG. 6 is a cross-sectional view for describing an internal structure of the sphygmomanometer cuff according to the first embodiment of the present invention.

FIG. 7 is a cross-sectional view showing a state in which the sphygmomanometer cuff according to the first embodiment of the present invention is worn at an upper arm.

FIG. 8 is an enlarged cross-sectional view of a region VIII shown in FIG. 7.

FIG. 9 is a cross-sectional view showing a first variant of the sphygmomanometer cuff according to the first embodiment of the present invention.

FIG. 10 is a cross-sectional view showing the first variant of the sphygmomanometer cuff according to the first embodiment of the present invention.

FIG. 11 is a cross-sectional view showing a second variant of the sphygmomanometer cuff according to the first embodiment of the present invention.

FIG. 12 is a cross-sectional view showing the second variant of the sphygmomanometer cuff according to the first embodiment of the present invention.

FIG. 13 is a developed view showing the second variant of the sphygmomanometer cuff according to the first embodiment of the present invention.

FIG. 14 is a developed view showing a third variant of the sphygmomanometer cuff according to the first embodiment of the present invention.

FIG. 15 is a cross-sectional view showing a fourth variant of the sphygmomanometer cuff according to the first embodiment of the present invention.

FIG. 16 is perspective view showing an outer appearance structure of a sphygmomanometer according to a second embodiment of the present invention.

FIG. 17 is perspective view showing an outer appearance structure of the sphygmomanometer according to the second embodiment of the present invention.

FIG. 18 is a cross-sectional view of a sphygmomanometer cuff according to the second embodiment of the present invention.

FIG. 19 is a cross-sectional view of the sphygmomanometer cuff according to the second embodiment of the present invention.

FIG. 20 is a cross-sectional view showing a first variant of the sphygmomanometer cuff according to the second embodiment of the present invention.

FIG. 21 is a cross-sectional view showing a second variant of the sphygmomanometer cuff according to the second embodiment of the present invention.

FIG. 22 is a cross-sectional view showing a third variant of the sphygmomanometer cuff according to the second embodiment of the present invention.

FIG. 23 is a cross-sectional view showing a fourth variant of the sphygmomanometer cuff according to the second embodiment of the present invention.

FIG. 24 is a cross-sectional view showing a fifth variant of the sphygmomanometer cuff according to the second embodiment of the present invention.

FIG. 25 is a cross-sectional view showing a sixth variant of the sphygmomanometer cuff according to the second embodiment of the present invention.

FIG. 26 is a schematic cross-sectional view of the sphygmomanometer cuff and the measuring site in a case where the cuff pressure is sufficiently high.

FIG. 27 is a schematic cross-sectional view of the sphygmomanometer cuff and the measuring site in a case where the cuff pressure is significantly low.

FIG. 28 is a graph of a pulse wave envelope showing one countermeasure for preventing lowering in the measurement accuracy by the pressure propagation loss.

FIG. 29 is a view showing a state in which a conventional sphygmomanometer cuff is wrapped around an upper arm, which is a measuring site.

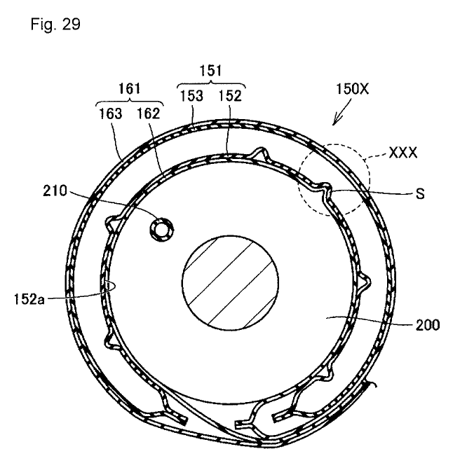

FIG. 30 is an enlarged cross-sectional view of a region XXX in FIG. 29.

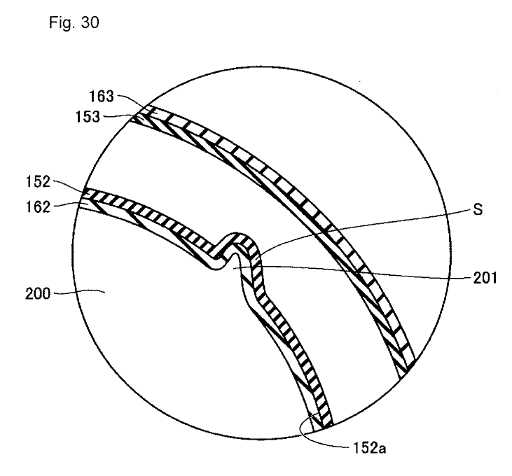

### DESCRIPTION OF REFERENCE SYMBOLS

100A, 100B: sphygmomanometer, 110: device main body, 114: display unit, 115: operation unit, 123: memory unit, 124: power supply unit, 125: oscillation circuit, 126: pump drive circuit, 127: valve drive circuit, 131: blood pressure measurement air system component, 132: pressure sensor, 133: inflation/contraction mechanism, 134: pump, 135: valve, 140: air tube, 150A to K, 150×: sphygmomanometer cuff, 151: air bladder, 152: inner sheet, 152a: compression acting surface, 153: outer sheet, 161: cover body, 162: inner peripheral side sheet portion, 162a: first sheet layer, 162b: second sheet layer, 162c: accommodation space, 163: outer peripheral side sheet portion, 164: surface fastener, 166a: joining portion, 166b: joining portion, 167: sutured portion (zigzag stitch), 168: shell, 168a: grip, 168b: unlock button, 171: cushion material, 171A to 171D: divided body, 171a to 171d: thick portion, 181: curler, 182: resin plate, 183: fabric, 200: upper arm, 210: artery.

### BEST MODE FOR CARRYING OUT THE INVENTION

Embodiments of the present invention will be described with reference to the drawings. In the embodiment described below, a sphygmomanometer mounted on an oscillometric type upper arm sphygmomanometer intended to have the upper arm as the measuring site, and a cuff thereof will be described by way of example.

### First Embodiment

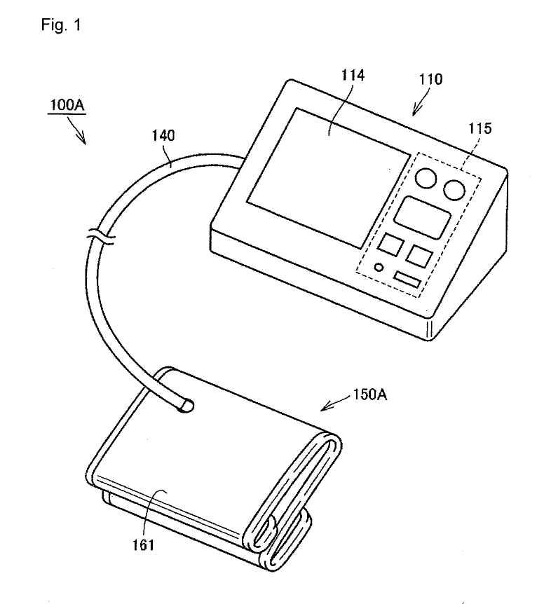

FIG. 1 is a perspective view showing an outer appearance of a sphygmomanometer according to a first embodiment of the present invention. First, the outer appearance structure of the sphygmomanometer according to the present embodiment will be described with reference to FIG. 1.

As shown in FIG. 1, a sphygmomanometer 100A according to the present embodiment mainly includes a device main body 110 and a cuff 150A. The device main body 110 has a display unit 114 and an operation unit 115. The display unit 114 visually displays the measurement result of a blood pressure value, the measurement result of a pulse rate, and the like using numerical values and graphs. A liquid crystal panel, or the like is used for the display unit 114. The operation unit 115 is arranged with a power button, a measurement start button, and the like.

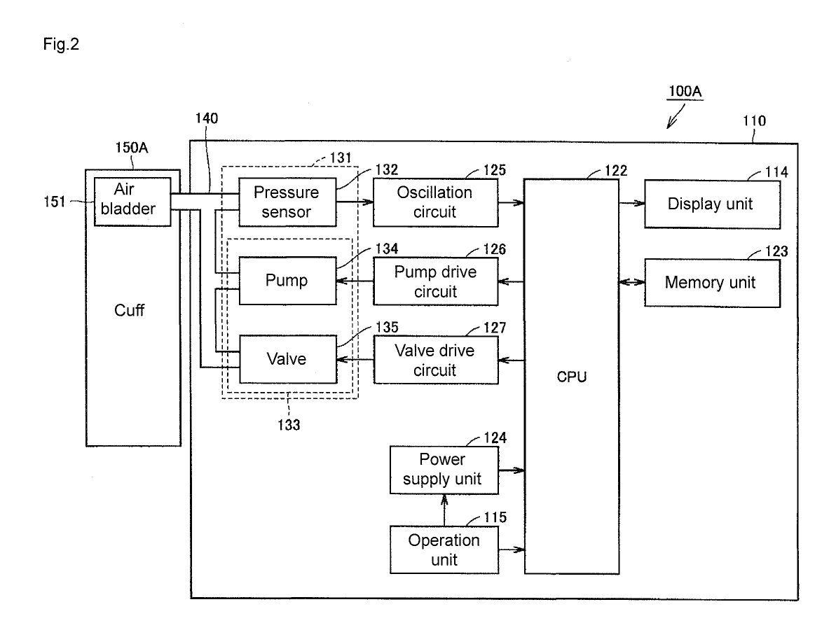

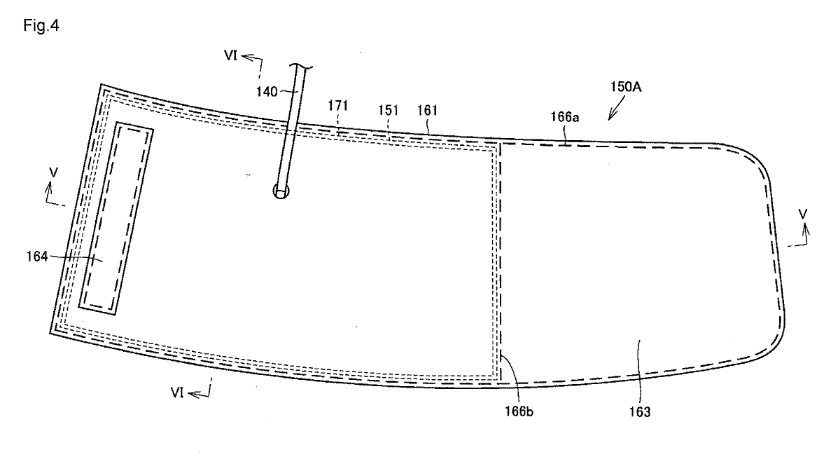

The cuff 150A is intended to be wrapped around the upper arm of the left arm or the upper arm of the right arm of a subject, and has a band-shaped outer shape. The cuff 150A includes an air bladder 151 (see FIG. 2, FIGS. 4 to 7, etc.) serving as a fluid bag for compressing the upper arm, and a cover body 161 (see FIGS. 4 to 7, etc.) serving as an exterior member for wrapping around and fixing the air bladder 151 to the upper arm. The air bladder 151 is accommodated in a space arranged inside the cover body 161. The detailed structure of the cuff 150A will be hereinafter described.

The cuff 150A and the device main body 110 are connected by an air tube 140 serving as a connection tube. The air tube 140 is a flexible tube, where one end is connected to a blood pressure measurement air system component 131 (see FIG. 2) arranged in the device main body 110, to be hereinafter described, and the other end is connected to the air bladder 151 of the cuff 150A described above.

FIG. 2 is a function block diagram showing a configuration of the sphygmomanometer according to the present embodiment. The configuration of the main function blocks of the sphygmomanometer according to the present embodiment will be described below with reference to FIG. 2.

As shown in FIG. 2, the blood pressure measurement air system component 131 for supplying or exhausting air to and from the air bladder 151 internally included in the cuff 150A through the air tube 140 is arranged inside the device main body 110 of the sphygmomanometer 100A. The blood pressure measurement air system component 131 includes a pressure sensor 132, which is a pressure detection unit, for detecting the pressure in the air bladder 151, and a pump 134 and a valve 135 serving as an inflation/contraction mechanism 133 for inflating or contracting the air bladder 151. An oscillation circuit 125, a pump drive circuit 126, and a valve drive circuit 127 are arranged inside the device main body 110 in relation to the blood pressure measurement air system component 131.

Furthermore, the device main body 110 is installed with a CPU (Central Processing Unit) 122 for controlling and monitoring each unit in a concentrated manner, a memory unit 123 for storing a program for causing the CPU 122 to perform a predetermined operation and various information such as measured blood pressure values, the display unit 114 for displaying various information including blood pressure measurement result, the operation unit 115 operated to input various instructions for measurement, and a power supply unit 124 for supplying power to the CPU 122 and each function block. The CPU 122 also functions as a blood pressure value calculation unit for calculating the blood pressure value.

The pressure sensor 132 detects the pressure in the air bladder 151 (hereinafter referred to as “cuff pressure”), and outputs a signal corresponding to the detected pressure to the oscillation circuit 125. The pump 134 supplies air to the air bladder 151. The valve 135 opens/closes when maintaining the pressure in the air bladder 151, or exhausting the air in the air bladder 151. The oscillation circuit 125 outputs a signal of an oscillating frequency corresponding to the output value of the pressure sensor 132 to the CPU 122. The pump drive circuit 126 controls the drive of the pump 134 based on a control signal provided from the CPU 122. The valve drive circuit 127 performs open/close control of the valve 135 based on a control signal provided from the CPU 122.

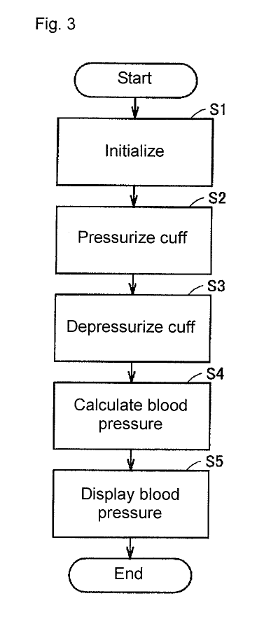

FIG. 3 is a flowchart showing a flow of a blood pressure measurement process of the sphygmomanometer according to the present embodiment. The flow of the blood pressure measurement process of the sphygmomanometer according to the present embodiment will be described with reference to FIG. 3. The program complying with the flowchart is stored in advance in the memory unit 123 shown in FIG. 2, and the CPU 122 reads out the program from the memory unit 123 and executes the same to perform the blood pressure measurement process.

As shown in FIG. 3, the sphygmomanometer 100A is initialized when the subject operates the operation button of the operation unit 115 of the sphygmomanometer 110A to turn ON the power (step S1). When a measureable state is obtained, the CPU 122 starts the drive of the pump 134 to gradually raise the cuff pressure of the air bladder 151 (step S2). When the cuff pressure reaches a predetermined level necessary for the blood pressure measurement in the process of gradually pressurizing the cuff pressure, the CPU 122 stops the pump 134, gradually opens the closed valve 135 to gradually exhaust the air of the air bladder 151, and gradually depressurizes the cuff pressure (step S3), and the cuff pressure is detected in the process of slow-speed depressurization process of the cuff pressure.

The CPU 122 then calculates the blood pressure value (systolic blood pressure value, diastolic blood pressure value) through a known procedure (step S4). Specifically, in the process of gradually depressurizing the cuff pressure, the CPU 122 extracts the pulse wave information based on the oscillating frequency obtained from the oscillation circuit 125. The blood pressure value is calculated from the extracted pulse wave information. After the blood pressure value is calculated in step S4, the calculated blood pressure value is displayed on the display unit 114 (step S5). The measurement method described above is based on a so-called depressurization measurement method of detecting the pulse wave when depressurizing the air bladder and calculating the blood pressure value, but it should be apparent that a so-called pressurization measurement method of detecting the pulse wave when pressurizing the air bladder and calculating the blood pressure value may be adopted.

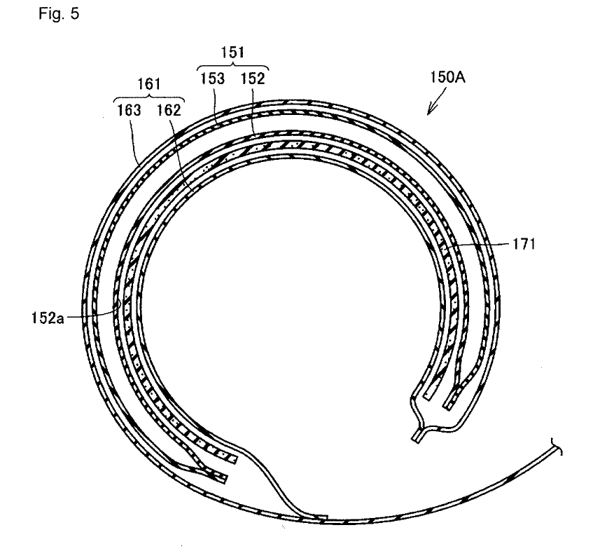

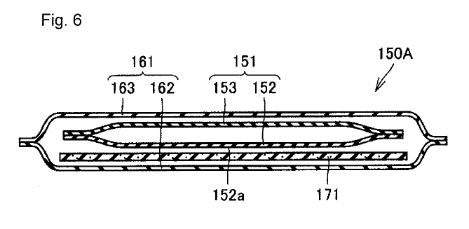

FIG. 4 is a view showing a state in which the sphygmomanometer cuff according to the present embodiment is developed. FIGS. 5 and 6 are cross-sectional views for describing an internal structure of the sphygmomanometer cuff according to the present embodiment, where FIG. 5 is a cross-sectional view taken along line V-V in FIG. 4, and FIG. 6 is a cross-sectional view taken along line VI-VI in FIG. 4. The sphygmomanometer cuff according to the present embodiment will be described in more detail below with reference to the drawings.

As shown in FIGS. 4 to 6, the sphygmomanometer cuff 150A according to the present embodiment mainly includes the air bladder 151, the cover body 161 internally including the air bladder 151, and a cushion material 171.

As shown in FIG. 4, the air bladder 151 has a substantially rectangular outer shape in a developed state, and is a bag-shaped member suitably formed using a resin sheet. As shown in FIGS. 5 and 6, the air bladder 151 is formed to a bag-shape by overlapping an inner sheet 152, which is to be positioned on the upper arm side when the cuff 150A is wrapped around the upper arm, and an outer sheet 153, which is to be positioned on the outer side than the inner sheet 152 when the cuff 150A is wrapped around the upper arm, and welding the peripheral edges, and is interiorly formed with an inflation/contraction space. The inflation/contraction space is connected to the air tube 140 so as to be pressurized/depressurized by the inflation/contraction mechanism 133. The main surface of the inner sheet 152 of the air bladder 151 that is to be positioned on the upper arm side when the sphygmomanometer cuff 150A is wrapped around the upper arm functions as a compression acting surface 152a for compressing the upper arm in the pressurizing state.

The material of the resin sheet configuring the air bladder 151 may be of any type as long as it excels in stretchability, and air does not leak out from the inflation/contraction space after welding. From such a standpoint, the suitable material of the resin sheet is ethylene-vinyl acetate copolymer (EVA), soft polyvinyl chloride (PVC), polyurethane (PU), polyamide (PA), raw rubber, or the like.

As shown in FIGS. 5 and 6, the cover body 161 is configured by an inner peripheral side sheet portion 162, which is to be positioned on the upper arm side when worn at the upper arm, and an outer peripheral side sheet portion 163, which is to be positioned on the side opposite to the upper arm with the air bladder 151 in between when worn at the upper arm, and is formed to a bag-shape by overlapping the inner peripheral side sheet portion 162 and the outer peripheral side sheet portion 163 and joining the peripheral edges.

The cover body 161 is suitably formed from a fabric of synthetic fiber such as polyamide (PA) and polyester, and welding, suturing, or the like is employed to join the inner peripheral side sheet portion 162 and the outer peripheral side sheet portion 163. The inner peripheral side sheet portion 162 of the cover body 161 is suitably configured by a member excelling in stretchability, and the outer peripheral side sheet portion 163 of the cover body 161 is suitably configured by a member which stretchability is inferior to the inner peripheral side sheet portion 162.

As shown in FIG. 4, the cushion material 171 is a compressible member of even thickness having a substantially rectangular outer shape in a developed state. As shown in FIGS. 4 to 6, the cushion material 171 is accommodated in a space formed in the interior of the cover body 161 along with the air bladder 151, and is interposed between the compression acting surface 152a of the air bladder 151 and the inner peripheral side sheet portion 162 of the cover body 161 so as to cover the entire surface of the compression acting surface 152a of the air bladder 151.

The cushion material 171 preferably has a high compressibility in the thickness direction, which compressibility is more suitably higher than the compressibility in the thickness direction of the inner peripheral side sheet portion 162. The hardness of the cushion material 171 may lead to the cause of lowering in accuracy of the blood pressure measurement, and thus is desirably smaller than or equal to the sponge hardness 20 (Asker C (SRIS-0101)). From such a standpoint, an independently foamed or simultaneously foamed sponge member made from rubber or synthetic resin can be used for the cushion material 171, and suitably, urethane foam, rubber sponge, or the like is particularly used. The thickness of the cushion material 171 is not particularly limited, but is suitably about greater than or equal to 1 mm and smaller than or equal to 15 mm.

As shown in FIG. 4, a surface fastener 164 is arranged on the outer peripheral surface at one end in the longitudinal direction of the cover body 161, and such a surface fastener 164 engages the inner peripheral surface at the other end in the longitudinal direction of the cover body 161. The surface fastener 164 is a locking portion for wrapping around and fixing the cuff 150A to the upper arm when the cuff 150A is worn at the upper arm, which is the measuring site.

As shown in FIG. 4, a joining portion 166a formed by joining the inner peripheral side sheet portion 162 and the outer peripheral side sheet portion 163 is positioned over the entire periphery at the peripheral edge of the cover body 161. A joining portion 166b extending in a width direction (i.e., direction orthogonal to the longitudinal direction) of the cover body 161 is also arranged at a predetermined position in the longitudinal direction of the cover body 161. The air bladder 151 and the cushion material 171 are accommodated in one space partitioned by the joining portion 166b out of the space in the interior of the cover body 161.

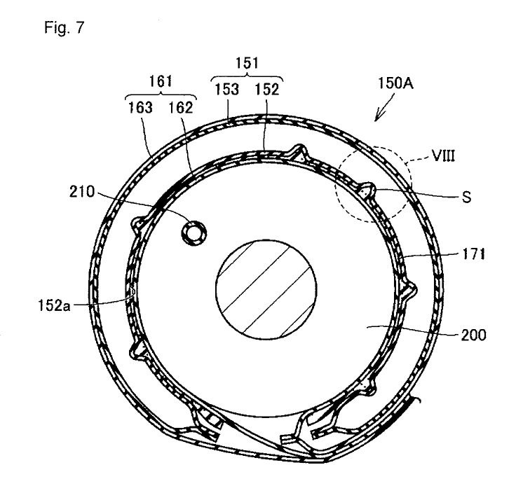

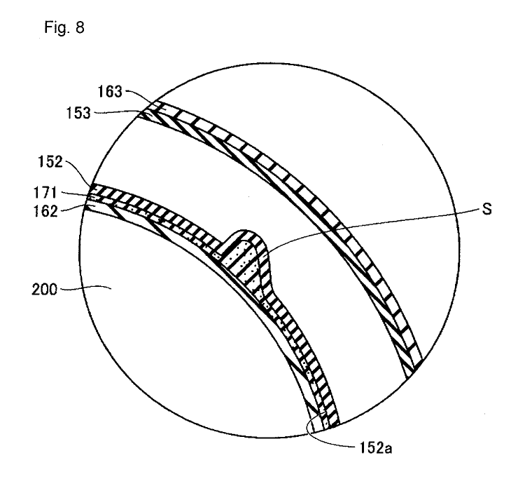

FIG. 7 is a cross-sectional view showing a state in which the sphygmomanometer cuff according to the present embodiment is worn at the upper arm, and FIG. 8 is an enlarged cross-sectional view of a region VIII shown in FIG. 7. The reason the occurrence of internal bleeding is prevented with the sphygmomanometer cuff according to the present embodiment will be described in detail below with reference to the drawings.

As shown in FIG. 7, the compression acting surface 152a of the air bladder 151 is positioned on the upper arm 200 side when the sphygmomanometer cuff 150A according to the present embodiment is worn at the upper arm 200. The inner peripheral side sheet portion 162 of the cover body 161, and the cushion material 171 are arranged in order from the upper arm 200 side between the compression acting surface 152a of the air bladder 151 and the upper arm 200.

When the air bladder 151 is inflated for blood pressure measurement, the inner sheet 152 of the air bladder 151 moves towards the upper arm 200 side with the inflation of the air bladder 151, and the compression acting surface 152a, which is the main surface on the upper arm 200 side, closely attaches to the cushion material 171. The cushion material 171 is then sandwiched by the air bladder 151 and the inner peripheral side sheet portion 162 of the cover body 161, and compressed in the thickness direction thereof. When the cushion material 171 is sufficiently compressed, the inner peripheral side sheet portion 162 of the cover body 161 also moves towards the upper arm 200 side, and closely attaches to the upper arm 200.

In this case, as shown in FIG. 8, the diameter of the inner sheet 152 itself of the air bladder 151 reduces and an extra portion produces in the inner sheet 152, where such an extra portion has nowhere to escape and thus moves to the outer side thereby forming wrinkles S at the compression acting surface 152a. The compression acting surface 152a of the inner sheet 152 of the air bladder 151 is closely attached to the cushion material 171, and thus the friction in between causes the cushion material 171 to be pulled into the valley portions of the wrinkles S at the inner sheet 151 of the air bladder 151 and enter the valley portions of the wrinkles S.

The wrinkles do not form at the inner peripheral side sheet portion 162 of the cover body 161 since the cushion material 171 is made from a material having excellent compressibility, as described above, and the frictional force is alleviated as part of the cushion material 171 elastically deforms. Thus, the close-attachment of the inner peripheral side sheet portion 162 and the upper arm 200 is not impaired, and obviously, part of the skin of the upper arm 200 will not be trapped in the wrinkles when measuring the blood pressure, and thus internal bleeding will not occur at the upper arm 200.

At the portion the wrinkles S are not formed, the cushion material 171 is sufficiently compressed by the air bladder 151 and the upper arm 200, and thus the compression force due to inflation of the air bladder 151 is sufficiently transmitted to the upper arm 200, and lack of compression force of the upper arm 200 due to the arrangement of the cushion material 171 does not arise.

Therefore, with the sphygmomanometer cuff 150A according to the present embodiment, even if wrinkles S are formed at the compression acting surface 152a of the air bladder 151 when measuring the blood pressure, the cushion material 171 mainly enters the valley portions of the wrinkles S, and there is no room for the skin to be caught at the valley portions of the winkles S. Therefore, the sphygmomanometer cuff in which the possibility of causing internal bleeding is greatly reduced is obtained. Furthermore, a sphygmomanometer in which internal bleeding does not occur at the measuring site in time of measurement is obtained with the sphygmomanometer equipped with the sphygmomanometer cuff.

As a secondary effect, the air bladder 151 is compressed from the outside by the restoring force of the cushion material 171 in exhausting air from the air bladder 151 during the blood pressure measurement or after the blood pressure measurement, and thus the air in the air bladder 151 can be rapidly pushed out, and an effect of more rapid blood pressure measurement can be expected. Moreover, since the cushion material 171 is arranged between the upper arm 200 and the air bladder 151 when the sphygmomanometer cuff 150A is worn, the feeling of pressure when compressing the upper arm 200 by the air bladder 151 becomes smooth, and the subject will not feel pain caused by the sudden compression. In addition, the air bladder 151 is protected since the compression acting surface 152a side of the air bladder 151 is covered by the cushion material 171.

In the sphygmomanometer cuff 150A according to the first embodiment described above, a case in which the cushion material 171 is not particularly fixed has been described by way of example, but a configuration of fixing the cushion material to the inner peripheral side sheet portion 162 of the cover body 161 or the inner sheet 152 of the air bladder 151 to prevent the cushion material 171 from shifting position or bending may be adopted. The fixing method for this case may be either method of welding or adhering, or a combination thereof when fixing the cushion material 171 to the inner peripheral side sheet portion 162 of the cover body 161. Any method of suturing, welding, or adhering, or a combination thereof may be used when fixing the cushion material 171 to the inner sheet 152 of the air bladder 151. If suturing is adopted, a zigzag stitch described in a second variant of the present embodiment, to be hereinafter described, is preferably used.

In the sphygmomanometer cuff 150A according to the present embodiment described above, a case in which the cushion material 171 is arranged between the compression acting surface 152a of the air bladder 151 and the inner peripheral side sheet portion 162 of the cover body 161 has been described by way of example. However, the cushion material 171 can exhibit the function of preventing internal bleeding as long as it is positioned on the inner peripheral side sheet portion 162 side than the compression acting surface 152a of the air bladder 151 when the sphygmomanometer cuff is worn. Variants of when the arrangement position of the cushion material 171 is changed will be described below.

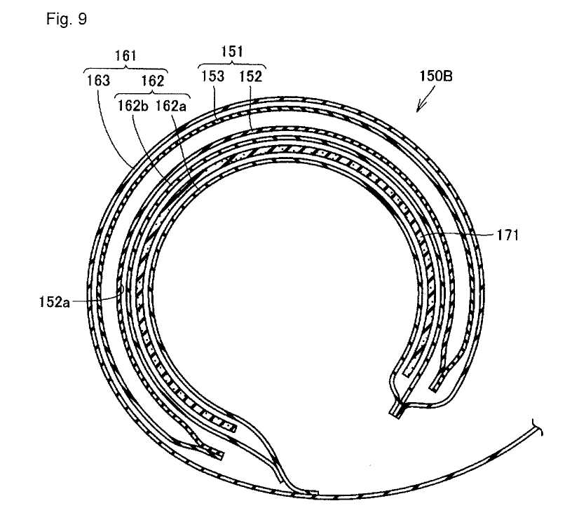

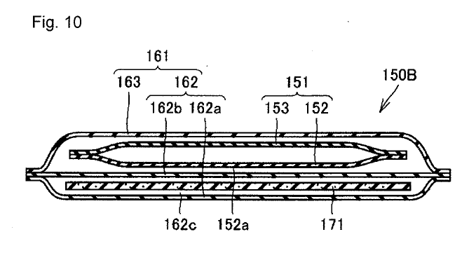

FIGS. 9 and 10 are cross-sectional views showing a first variant of the sphygmomanometer cuff according to the present embodiment. In a sphygmomanometer cuff 150B shown in FIGS. 9 and 10, the inner peripheral side sheet portion 162 of the cover body 161 has a two-layer structure including a first sheet layer 162a and a second sheet layer 162b, where an accommodation space 162c is formed between the first sheet layer 162a and the second sheet layer 162b. A configuration in which the cushion material 171 is arranged in the accommodation space 162c is adopted.

Even if configured as above, the cushion material 171 and the second sheet layer 162b of the inner peripheral side sheet portion 162 mainly enter the valley portions of the wrinkles when wrinkles are formed at the compression acting surface 152a of the air bladder 151 when measuring the blood pressure, and thus there is no room for the skin to be caught at the valley portions of the wrinkles. Therefore, a sphygmomanometer cuff in which the possibility of causing internal bleeding is greatly reduced is obtained.

In the sphygmomanometer cuff 1506 according to the first variant, a case in which the cushion material 171 is not particularly fixed has been described by way of example, but a configuration in which the cushion material is fixed to the inner peripheral side sheet portion 162 to prevent the cushion material 171 from shifting position or bending may be adopted. In this case, the cushion material 171 may be attached to the first sheet layer 162a or/and the second sheet layer 162b of the inner peripheral side sheet portion 162. The fixing method for this case may be any method of suturing, welding, or adhering, or a combination thereof. If suturing is adopted, a zigzag stitch described in a second variant, to be hereinafter described, is preferably used. Furthermore, an advantage in that the stitches are not exposed at the surface of the sphygmomanometer cuff 1508 can be obtained by suturing the cushion material 171 to the second sheet layer 162b when adopting suturing.

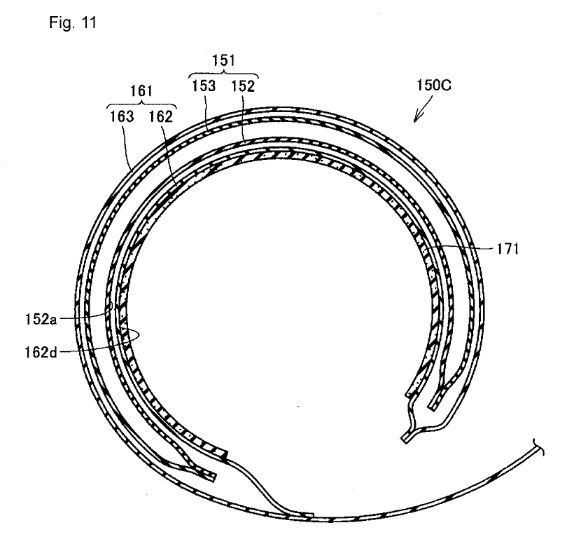

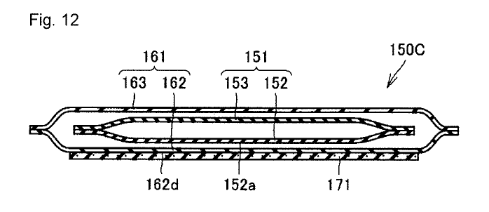

FIGS. 11 and 12 are cross-sectional views each showing a second variant of the sphygmomanometer cuff according to the present embodiment. In a sphygmomanometer cuff 150C shown in FIGS. 11 and 12, a configuration in which the cushion material 171 is arranged so as to cover the main surface on the side opposite to the compression acting surface 152a of the inner peripheral side sheet portion 162 of the cover body 161 (i.e., exposed surface of the cover body 161 that faces the upper arm side when the sphygmomanometer cuff 150C is worn) is adopted.

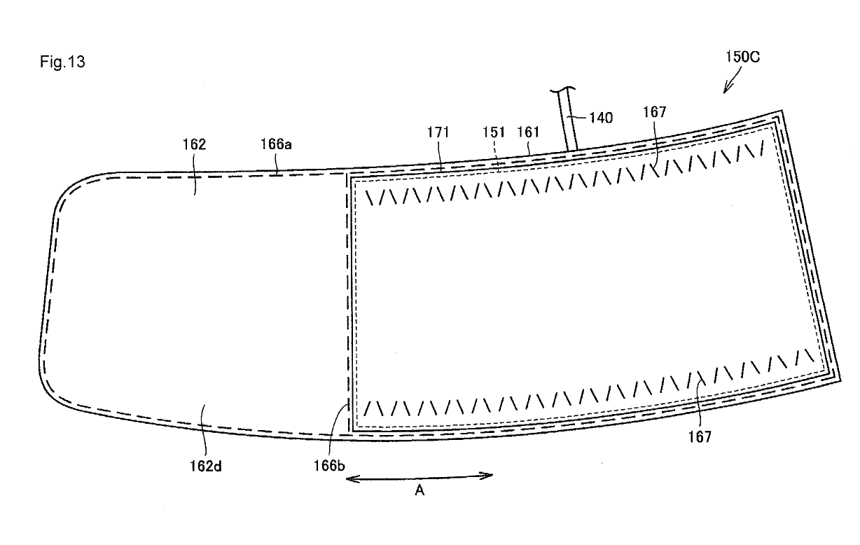

When configured in such a manner, the cushion material 171 needs to be fixed to the cover body 161. FIG. 13 is a view showing one example of fixing the cushion material to the cover body in the present variant. As shown in FIG. 13, in the sphygmomanometer cuff 150C according to the present variant, the cushion material 171 is attached to the inner peripheral side sheet portion 162 of the cover body 161 along the longitudinal direction (direction of arrow A shown in the figure) of the sphygmomanometer cuff 150C by suturing. The suturing method is a so-called zigzag stitch as shown with a reference numeral 167 in the figure. Through the use of the zigzag stitch, the cushion material 171 can also move following the stretching in the longitudinal direction of the cover body 161, and thus the cushion material 171 does not inhibit the stretching of the cover body 161.

Even if configured as above, the cushion material 171 and the inner peripheral side sheet portion 162 mainly enter the valley portions of the wrinkles when wrinkles are formed at the compression acting surface 152a of the air bladder 151 when measuring the blood pressure, and thus there is no room for the skin to be caught at the valley portions of the wrinkles. Therefore, a sphygmomanometer cuff in which the possibility of causing internal bleeding is greatly reduced is obtained.

In the sphygmomanometer cuff 150C according to the second variant, a case in which the cushion material 171 is attached to the inner peripheral side sheet portion 162 by suturing has been described by way of example, but the cushion material 171 may be attached to the inner peripheral side sheet portion 162 by welding or adhering.

In the present embodiment, the first variant, and the second variant, a case in which the cushion material 171 is arranged so as to cover the entire surface of the compression acting surface 152a of the air bladder 151 has been described by way of example, but the cushion material 171 may cover only part of the compression acting surface 152a of the air bladder 151, in which case the cushion material 171 may be configured by a plurality of divided bodies. In this case, the cushion material or the divided body thereof is preferably arranged so as to cover the portion where the wrinkle is likely to form at the air bladder 151. A variant of one example of when configured in such a manner will be described below.

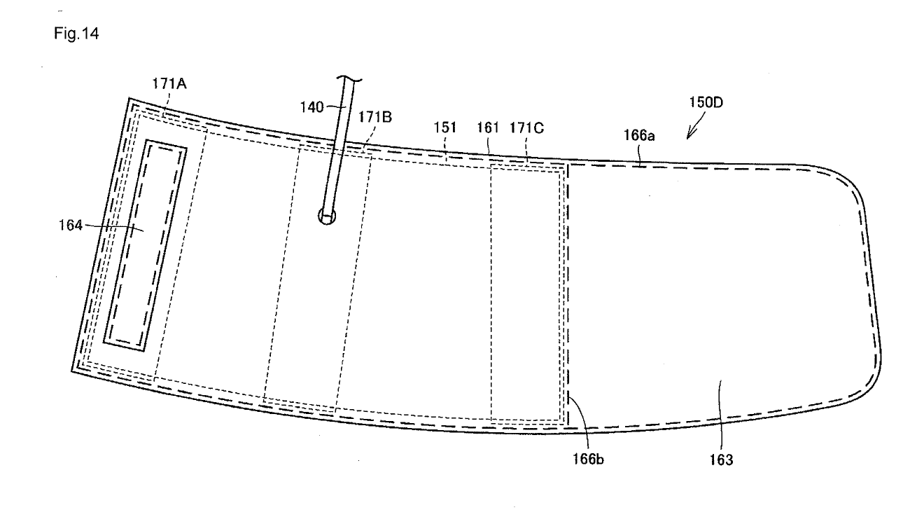

FIG. 14 is a developed view showing a third variant of the sphygmomanometer cuff according to the present embodiment. As shown in FIG. 14, in a sphygmomanometer cuff 150D according to the present variant, the cushion material is configured by a plurality of divided bodies 171A to 171C, and each divided body 171A to 171C is arranged so as to respectively cover a predetermined position of the compression acting surface 152a of the air bladder 151. Specifically, as shown in FIG. 14, the divided body 171A is arranged to cover the vicinity of one end in the longitudinal direction of the rectangular air bladder 151, the divided body 171B is arranged to cover the vicinity of a nipple portion to where the air tube 140 is connected of the air bladder 151, and the divided body 171C is arranged to cover the vicinity of the other end in the longitudinal direction of the rectangular air bladder 151.

According to such a configuration, the divided body 171 of the cushion material is arranged between the upper arm and the air bladder 151 at both ends in the longitudinal direction of the air bladder and the vicinity of the nipple portion, which are portions where wrinkles particularly easily form, and thus the cushion material 171 and the inner peripheral side sheet portion 162 mainly enter the valley portions of the wrinkles and there is no room for the skin to be caught at the valley portions of the wrinkles. Therefore, a sphygmomanometer cuff in which the possibility of causing internal bleeding is greatly reduced is obtained.

In the present embodiment, and the first variant to the third variant, a sphygmomanometer cuff in which only the air bladder 151 and the cushion material 171 are accommodated inside the cover body 161 has been described by way of example, but a configuration including other internal structures may be adopted. One example will be described below.

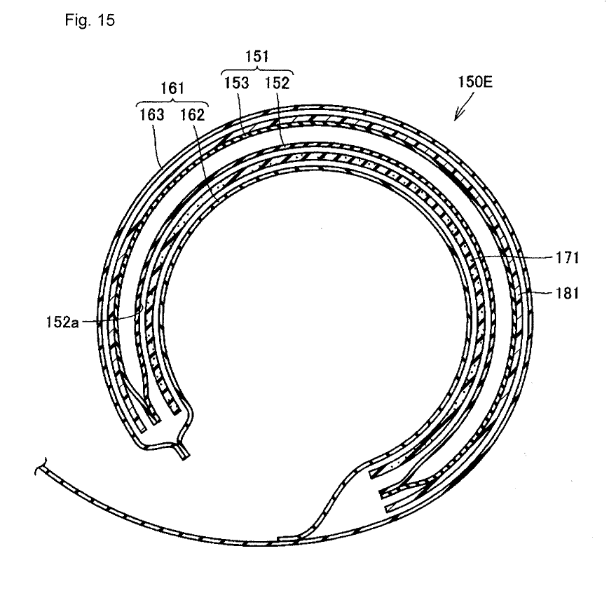

FIG. 15 is a cross-sectional view showing a fourth variant of the sphygmomanometer cuff according to the present embodiment. As shown in FIG. 15, in a sphygmomanometer cuff 150E according to the present variant, a curler (curved elastic plate) 181 serving as a bias portion for biasing the air bladder 151 towards the inner side is arranged. The curler 181 is formed to a cylindrical shape so as to fit to the upper arm, and is made from a flexible member so as to be elastically deformable in the radial direction when wounded to an annular-shape. The curler 181 is arranged inside the cover body 161 and on the outer side of the air bladder 151.

The curler 181 is adhered/fixed to the outer peripheral surface of the air bladder 151 by an adhesive member such as a double-sided tape (not shown). The curler 181 is configured to lie along the upper arm by maintaining its annular state, and enables the cuff 150E to be easily worn at the upper arm by the subject and biases the air bladder 151 towards the upper arm side when the cuff 150E is worn at the upper arm. The curler 181 is made from resin material such as polypropylene (PP) to express sufficient elastic force.

Even if configured as above, the cushion material 171 mainly enters the valley portions of the wrinkles when wrinkles are formed at the compression acting surface 152a of the air bladder 151 when measuring the blood pressure, and thus there is no room for the skin to be caught at the valley portions of the wrinkles. Therefore, a sphygmomanometer cuff in which the possibility of causing internal bleeding is greatly reduced is obtained.

### Second Embodiment

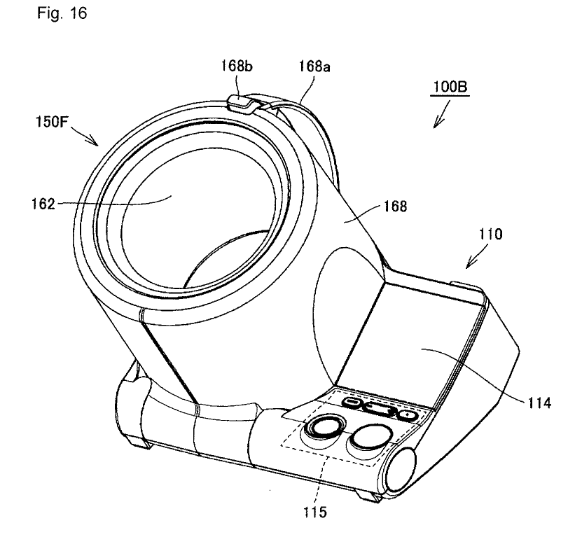

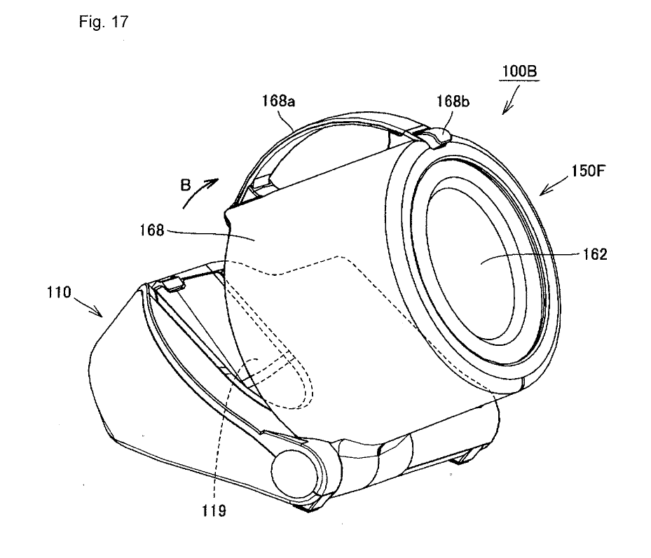

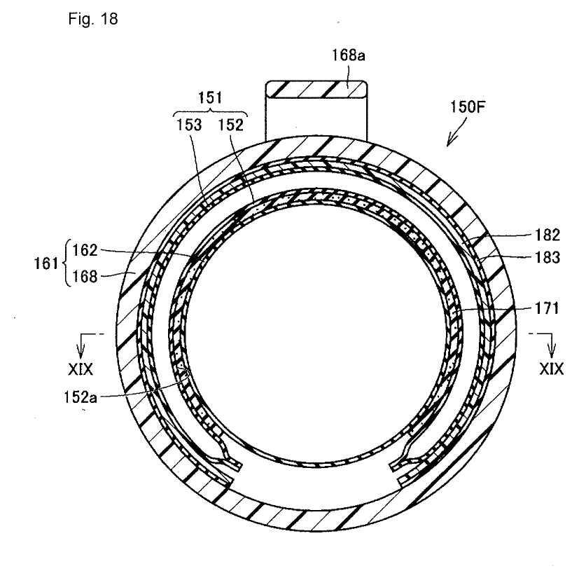

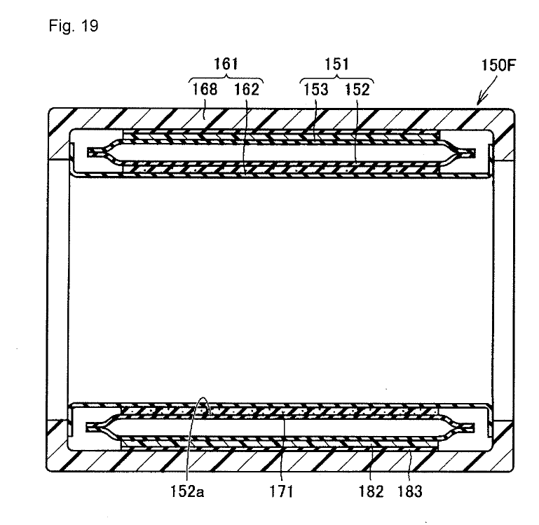

FIGS. 16 and 17 are perspective views showing an outer appearance structure of a sphygmomanometer according to a second embodiment of the present invention. FIGS. 18 and 19 are cross-sectional views of a sphygmomanometer cuff according to the present embodiment. The cross-section of the sphygmomanometer cuff shown in FIG. 19 is a cross-sectional view taken along line XIX-XIX shown in FIG. 18. The function blocks and the measurement flow of the sphygmomanometer 100B according to the present embodiment conform to those of the sphygmomanometer 100A according to the first embodiment, and thus the description thereof will not be repeated.

First, the outer appearance structure of the sphygmomanometer according to the present embodiment will be described with reference to FIGS. 16 and 17. As shown in FIGS. 16 and 17, the sphygmomanometer 1008 according to the present embodiment mainly includes the device main body 110 mounted on a mounting surface of a desk, or the like, and a cuff 150F serving as an upper arm inserting section having a hollow portion to which the upper arm of the subject is inserted.

The device main body 110 has the display unit 114 and the operation unit 115. The display unit 114 visually displays the measurement result of the blood pressure value, the measurement result of the pulse rate, and the like using numerical values and graphs. A liquid crystal panel, or the like is used for the display unit 114. The operation unit 115 is arranged with a power button, a measurement start button, and the like. An elbow placing section 119 for the subject to place the elbow when taking a measurement position is arranged at a predetermined position on the upper surface of the device main body 110 adjacent to the operation unit 115 and the display unit 114, as shown in FIG. 17. The elbow placing section 119 is formed, for example, by forming a recessed portion at the upper surface of the device main body 110.

The cuff 150F serving as the upper arm inserting section includes a shell 168 having a substantially cylindrical outer shape. A grip 168a gripped by the subject to turnably move the cuff 150F serving as the upper arm inserting section is arranged at a predetermined position on the outer peripheral surface of the shell 168. An unlock button 168b used to turnably move the cuff 150F accommodated on the device main body 110 is arranged near the grip 168a. A detailed structure (in particular, internal structure) of the cuff 150F will be hereinafter described.

The cuff 150F is coupled to be freely turnable in the up and down direction with respect to the device main body 110 by a turnably coupling mechanism including a turning shaft. Specifically, the device main body 110 and the cuff 150F are coupled in a freely turning manner in a direction of an arrow B in the figure by the turning shaft arranged in the device main body 110 closer to the front end positioned on the subject side.

The internal structure of the sphygmomanometer cuff according to the present embodiment will be described with reference to FIGS. 18 and 19. As shown in FIGS. 18 and 19, the cuff 150F according to the present embodiment mainly includes the air bladder 151 serving as the fluid bag, and the shell 168 and the inner peripheral side sheet portion 162 serving as the cover body internally including the air bladder 151, and the cushion material 171.

The air bladder 151 has a substantially rectangular outer shape in the developed state, and is a bag-shaped member suitably formed using a resin sheet. The air bladder 151 is formed to a bag-shape by overlapping the inner sheet 152, which is to be positioned on the upper arm side when the upper arm is inserted to the hollow portion of the cuff 150F, and the outer sheet 153, which is to be positioned on the outer side than the inner sheet 152, and welding the peripheral edges, and is interiorly formed with an inflation/contraction space. The inflation/contraction space is connected to the air tube (not shown) so as to be pressurized/depressurized by the inflation/contraction mechanism (not shown) arranged in the device main body 110. The main surface of the inner sheet 152 of the air bladder 151 that is to be positioned on the upper arm side when the upper arm is inserted to the hollow portion of the cuff 150F functions as the compression acting surface 152a for compressing the upper arm in the pressurizing state.

The cover body 161 is configured by the inner peripheral side sheet portion 162, which is the inner fabric to be positioned on the upper arm side when worn at the upper arm, and the shell 168 configuring the casing of the cuff 150F serving as the upper arm inserting section, where the air bladder 151 described above is accommodated therein.

The cushion material 171 is a compressible member of even thickness having a substantially rectangular outer shape in a developed state. The cushion material 171 is accommodated in the space formed in the interior of the cover body 161 along with the air bladder 151, and is interposed between the compression acting surface 152a of the air bladder 151 and the inner peripheral side sheet portion 162 of the cover body 161 so as to cover the entire surface of the compression acting surface 152a of the air bladder 151. The cushion material 171 is arranged to cover the entire surface of the compression acting surface 152a of the air bladder 151.

The material and the shape similar to those described in the first embodiment can be used for the air bladder 151, the inner peripheral side sheet portion 161, and the cushion material 171.

In addition to the air bladder 151 and the cushion material 171, a resin plate 182 having a relatively large rigidity, which is a shape maintaining member, positioned on the outer side of the air bladder 151 to maintain the shape of the air bladder 151 of small rigidity, and a fabric 183, which is a low friction member, positioned on the outer side of the resin plate 182 and contacting the inner peripheral surface side of the shell 168 are arranged in the interior of the cover body 161 including the shell 168 and the inner peripheral side sheet portion 162.

Therefore, effects similar to the first embodiment are obtained even if the sphygmomanometer and the sphygmomanometer cuff are configured as above. In other words, the cushion material 171 mainly enters the valley portions of the wrinkles when wrinkles are formed at the compression acting surface 152a of the air bladder 151 when measuring the blood pressure, and thus there is no room for the skin to be caught at the valley portions of the wrinkles. Therefore, a sphygmomanometer cuff in which the possibility of causing internal bleeding is greatly reduced and the sphygmomanometer equipped with the same are obtained.

In the sphygmomanometer cuff 150F according to the present embodiment, a case in which the cushion material 171 is arranged between the compression acting surface 152a of the air bladder 151 and the inner peripheral side sheet portion 162 of the cover body 161 has been described by way of example. However, the function of preventing internal bleeding is exhibited as long as the cushion material 171 is positioned on the inner peripheral side sheet portion 162 side than the compression acting surface 152a of the air bladder 151 when the sphygmomanometer cuff is worn. Variants of when the arrangement position of the cushion material 171 is changed will be described below.

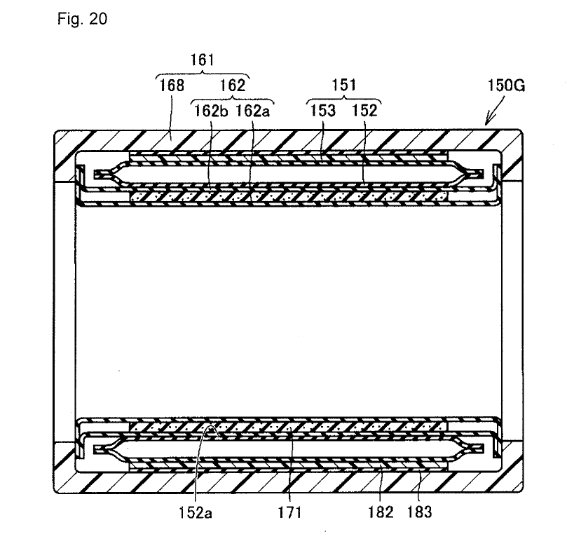

FIG. 20 is a cross-sectional view showing a first variant of the sphygmomanometer cuff according to the present embodiment. In a sphygmomanometer cuff 150G shown in FIG. 20, the inner peripheral side sheet portion 162 of the cover body 161 has a two-layer structure including the first sheet layer 162a and the second sheet layer 162b, where the accommodation space 162c is formed between the first sheet layer 162a and the second sheet layer 162b. A configuration in which the cushion material 171 is arranged in the accommodation space 162c is adopted.

Even if configured as above, the cushion material 171 and the second sheet layer 162b of the inner peripheral side sheet portion 162 mainly enter the valley portions of the wrinkles when wrinkles are formed at the compression acting surface 152a of the air bladder 151 when measuring the blood pressure, and thus there is no room for the skin to be caught at the valley portions of the wrinkles. Therefore, a sphygmomanometer cuff in which the possibility of causing internal bleeding is greatly reduced is obtained.

Although illustration will be omitted, the cushion material 171 may obviously be attached to the exposed surface of the inner peripheral side sheet portion 162 of the cover body 161, similar to the third variant of the first embodiment, where internal bleeding can be prevented in this case as well.

In the present embodiment and the first variant thereof, a case of using a sheet-like cushion material having an even thickness has been described by way of example, but the thickness and the shape may be variously changed. Cases in which the thickness, the shape, and the like of the cushion material are variously changed will be described below as variants.

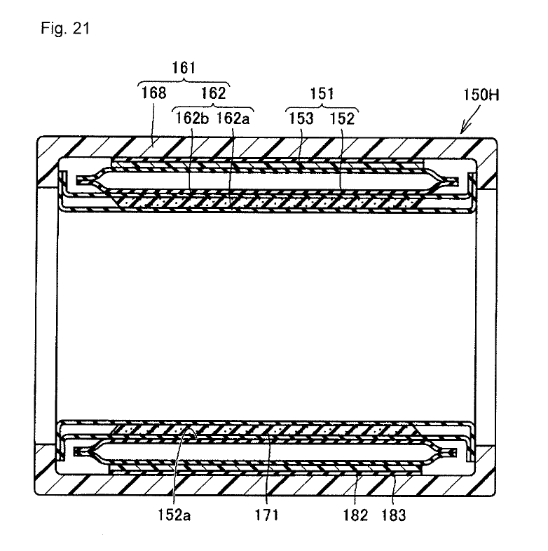

FIG. 21 is a cross-sectional view showing a second variant of the sphygmomanometer cuff according to the present embodiment. The present variant is one example in a case where the thickness of the cushion material is changed in the axial direction of the cuff. As shown in FIG. 21, in a sphygmomanometer cuff 150H according to the present variant, the shape of the cushion material 171 at the portion corresponding to the vicinity of both openings of the hollow portion to which the upper arm is inserted has a tapered shape. That is, the thickness of the cushion material gradually thickens from the entrance side towards the exit side at the opening on the entrance side of the hollow portion to be positioned on the subject side, and the thickness of the cushion material gradually thins from the entrance side towards the exit side at the opening on the exit side of the hollow portion to be positioned on the far side. According to such a configuration, internal bleeding does not occur at the upper arm when measuring the blood pressure, and furthermore, the upper arm can be smoothly placed in and out from the opening when inserting and removing the upper arm.

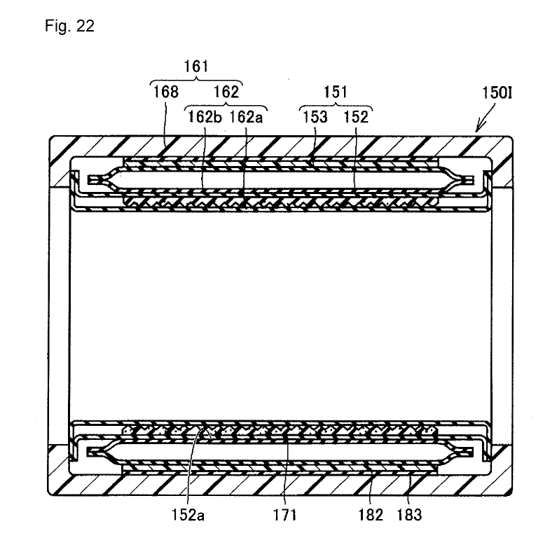

FIG. 22 is a cross-sectional view showing a third variant of the sphygmomanometer cuff according to the present embodiment. The present variant is one example in a case where the thickness of the cushion material is changed in the axial direction and the peripheral direction of the cuff. As shown in FIG. 22, in a sphygmomanometer cuff 150I according to the present variant, a plurality of bumps is formed on the main surface of the cushion material 171 to be positioned on the upper arm side when the upper arm is inserted to the hollow portion of the cuff 150I. According to such a configuration, internal bleeding does not occur at the upper arm when measuring the blood pressure, and furthermore, friction when inserting and removing the upper arm can be reduced, and the upper arm can be smoothly placed in and out from the opening.

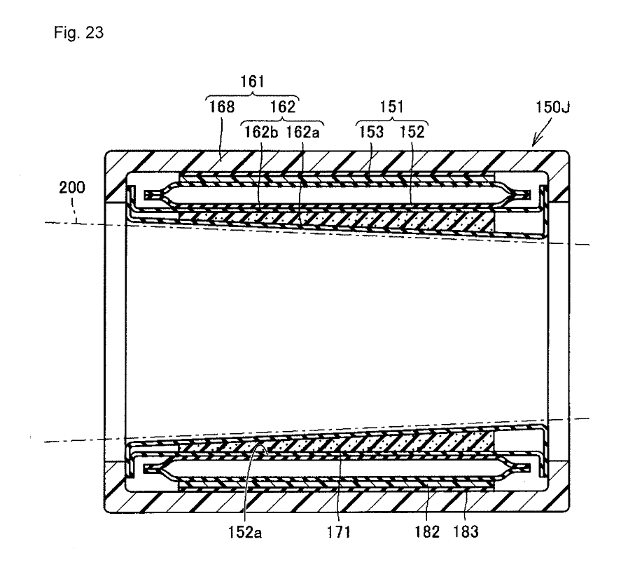

FIG. 23 is a cross-sectional view showing a fourth variant of the sphygmomanometer cuff according to the present embodiment. The present variant is one example in a case where the thickness of the cushion material is changed in the axial direction of the cuff. As shown in FIG. 23, in a sphygmomanometer cuff 150J according to the present variant, the shape of the cushion material 171 is formed to a tapered shape over the entire hollow portion to which the upper arm is inserted. That is, the thickness of the cushion material is gradually thinned from the opening on the entrance side to the opening on the exit side of the hollow portion to be positioned on the subject side. According to such a configuration, internal bleeding does not occur at the upper arm when measuring the blood pressure, and furthermore, the cuff 150J fits to the upper arm inserted to the hollow portion and formation of wrinkles is prevented in that sense.

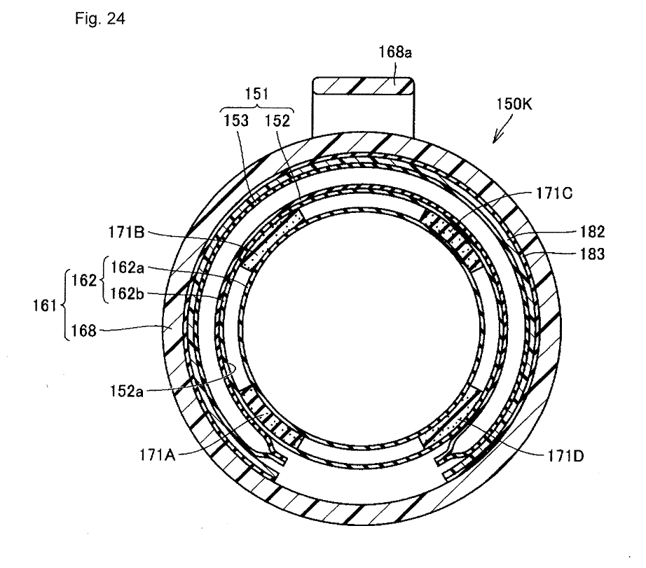

FIG. 24 is a cross-sectional view showing a fifth variant of the sphygmomanometer cuff according to the present embodiment. The present variant is one example in a case where the cushion material is arranged in a divided manner. Similar to the third variant of the first embodiment, in a sphygmomanometer cuff 150K according to the present variant, the cushion material is configured by a plurality of divided bodies 171A to 171D, and each divided body 171A to 171D is arranged so as to respectively cover the predetermined position of the compression acting surface 152a of the air bladder 151, as shown in FIG. 24. The internal bleeding can be effectively prevented with small amount of material by arranging the divided bodies 171A to 171D of the cushion material so as to cover the portion where wrinkles easily form at the air bladder 151.

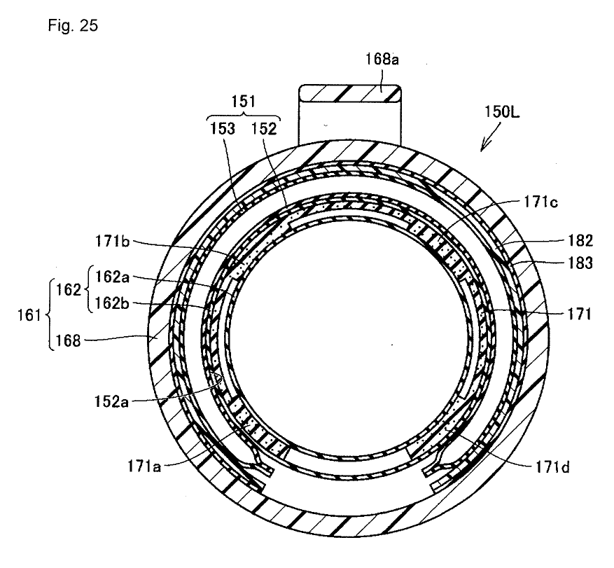

FIG. 25 is a cross-sectional view showing a sixth variant of the sphygmomanometer cuff according to the present embodiment. The present variant is one example in a case where the thickness of the cushion material is changed in the peripheral direction of the cuff. As shown in FIG. 25, in a sphygmomanometer cuff 150L according to the present variant, the thickness of the cushion material 171 at the portion where wrinkles easily form of the air bladder 151 in the peripheral direction of the cuff 150L is thickened as thick portions 171a to 171d, and the thickness of the cushion material 171 at the portion where wrinkles are less likely to form of the air bladder 151 in the peripheral direction of the cuff 150L is thinned as thin portion. According to such a configuration, the internal bleeding can be effectively prevented with small amount of material, similar to the fifth variant of the present embodiment.

In the first and second embodiments as well as the variants thereof, the cushion material is positioned between the compression acting surface of the air bladder and the measuring site of the living body when the sphygmomanometer cuff is worn. When measuring the blood pressure, the cushion material is sufficiently compressed in the thickness direction, so that the compression force on the measuring site by the air bladder does not lower. However, if the cuff pressure is significantly low even though the cushion material is sufficiently soft, or if the cushion material is relatively hard, the pressure may be attenuated by the cushion material in the process of the arterial pressure being transmitted to the air bladder. The reason for occurrence of such a problem will be described below with reference to the drawings.

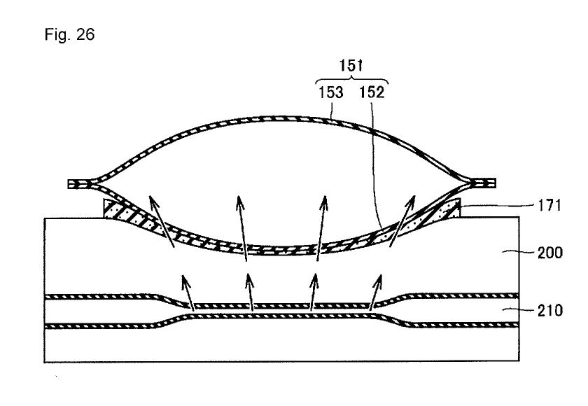

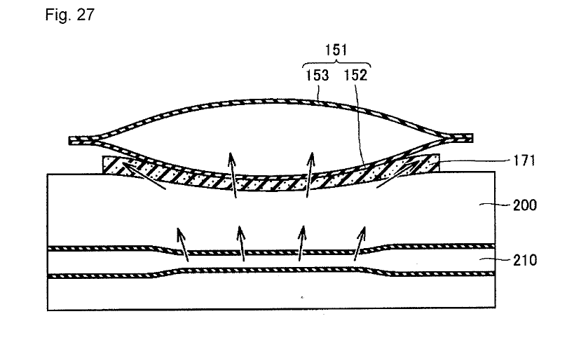

FIGS. 26 and 27 are cross-sectional views schematically showing a state in which the sphygmomanometer cuff using a sufficiently soft cushion material is attached to the measuring site of the living body, where FIG. 26 is a view showing a case in which the cuff pressure is sufficiently high, and FIG. 27 is a view showing a case in which the cuff pressure is significantly low. Such cross-sectional views are cross-sectional views taken along an extending direction of the artery, and the illustration of the inner peripheral side sheet portion of the cover body positioned between the air bladder and the living body under normal circumstances is omitted.

As shown in FIG. 26, if the cuff pressure is high enough to compress the cushion material when measuring the blood pressure value, the cushion material 171 is sufficiently compressed in the thickness direction thereof, and the internal pressure of the artery 210 (arterial pressure) is transmitted to the air bladder 151 without being lost through the arterial wall, the living body tissue, and the cushion material 171. As shown in FIG. 27, however, if the cuff pressure is significantly low when measuring the blood pressure value, part of the arterial pressure is absorbed and attenuated by the cushion material 171, and then transmitted to the air bladder 151 in the process of the arterial pressure being transmitted to the air bladder. Generally, the systolic blood pressure value (SYS) is measured when the cuff pressure is high pressure, and the diastolic blood pressure value (DIA) is measured when the cuff pressure is low pressure in the sphygmomanometer. Therefore, if the cuff is not sufficiently strongly wrapped around the measuring site when worn or if used by a subject whose diastolic blood pressure value is significantly low, the pressure propagation loss by the cushion material occurs, and the measured diastolic blood pressure value may be detected higher than the actual diastolic blood pressure value. Similar problems arise when the hardness of the cushion material is relatively hard.

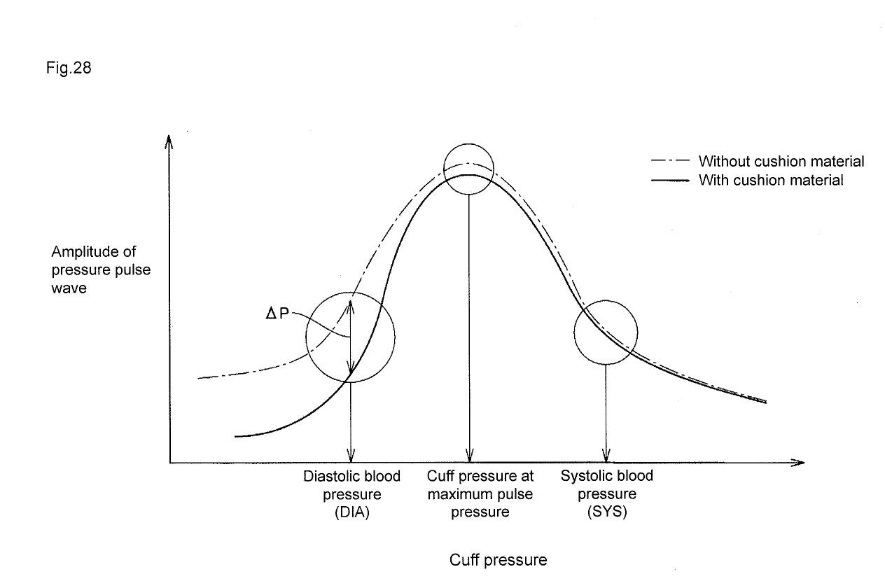

One countermeasure for preventing lowering in the measurement accuracy by the pressure propagation loss will be described below. FIG. 28 is a view for describing one countermeasure for preventing lowering in the measurement accuracy by the pressure propagation loss, and is a graph of a pulse wave envelope showing a relationship between the cuff pressure and the amplitude of the pressure pulse wave in time of blood pressure measurement. In FIG. 28, the cuff pressure is shown on the horizontal axis, and the amplitude of the pressure pulse wave is shown on the vertical axis. The ΔP shown in FIG. 28 is the pressure difference corresponding to the pressure propagation loss of the arterial pressure that becomes the cause of calculation of the diastolic blood pressure value.

This countermeasure references the cuff pressure in a state where the cushion material is sufficiently compressed and the pressure propagation loss by the cushion material is presumed to be small to a negligible extent, and compares with a predefined value to correct the diastolic blood pressure value, thereby preventing lowering in the measurement accuracy.

Specifically, for example, when the determining condition of the diastolic blood pressure value A_{DIA} is calculated from a maximum pulse wave amplitude value A_{MAX}, the relationship thereof is as expressed in the following equation (1).

A_{DIA}=α×A_{MAX} (1)

Here, α is a coefficient derived from great number of data.

Assuming correction is performed when the maximum pulse wave amplitude value A_{mAx} is smaller than 80 mmHg, the calculation formula of the diastolic blood pressure value A_{DIA} of when performing correction is as expressed in the following equation (2).

A_{DIA}=α×A_{MAX}×(1−(80−P)/80) (2)

Here, P is a cuff pressure value at the time point the maximum pulse wave amplitude value is observed.

If such countermeasure is adopted, the error of the diastolic blood pressure value that occurs with the pressure propagation loss can be corrected through correction, whereby a more accurate blood pressure value measurement becomes possible. In the specific example described above, one example in the case where the diastolic blood pressure value A_{DIA} is determined by the maximum pulse wave amplitude value A_{MAX} is shown, but correction can be applied through a similar method even when the diastolic blood pressure value A_{DIA} is determined by the systolic blood pressure value A_{SYS}.

In the first and second embodiments as well as the variants thereof described above, the sphygmomanometer cuff intended to be worn at the upper arm and the sphygmomanometer equipped with the same have been described by way of example, but the present invention may be applied to the sphygmomanometer cuff intended to be worn at the wrist and the sphygmomanometer equipped with the same.

It should be apparent that the characteristic configurations described in the first and the second embodiments as well as the variants thereof may be implemented by being combined with each other.

In the first and the second embodiments as well as the variants thereof, a case in which the present invention is applied to the oscillometric type sphygmomanometer cuff has been described by way of example, but it should be apparent that the present invention may be applied to a Korotkoff type sphygmomanometer cuff.

Each embodiment disclosed herein is illustrative in all aspects, and is not restrictive. The technical scope of the invention is defined by the claims, and encompasses meanings equivalent to the description of the claims and all modifications made within the scope.

1. A sphygmomanometer cuff used by being wrapped around a living body; the sphygmomanometer cuff comprising:
a fluid bag, which inflates/contracts by in/out of fluid, including a compression acting surface positioned on the living body side when the sphygmomanometer cuff is wrapped around the living body;
a cover body, which internally includes the fluid bag, including an inner peripheral side sheet portion positioned on the living body side when the sphygmomanometer cuff is wrapped around the living body; and
a cushion material positioned on the inner peripheral side sheet portion side than the compression acting surface, and compressible in a direction parallel to a thickness direction of the inner peripheral side sheet portion,
wherein the cushion material is positioned so as to cover the entire surface of the compression acting surface.

2. The sphygmomanometer cuff according to claim 1, wherein the cushion material has compressibility higher than compressibility in the thickness direction of the inner peripheral side sheet portion in the direction parallel to the thickness direction of the inner peripheral side sheet portion.

3. The sphygmomanometer cuff according to claim 1, wherein the cushion material is an independently foamed or simultaneously foamed sponge member made of rubber or synthetic resin.

4. The sphygmomanometer cuff according to claim 1, wherein the cushion material is positioned between the compression acting surface and the inner peripheral side sheet portion.

5. The sphygmomanometer cuff according to claim 1, wherein
the inner peripheral side sheet portion has a two-layer structure with a space inside; and
the cushion material is positioned in the space.

6. The sphygmomanometer cuff according to claim 1, wherein the cushion material is positioned on a side opposite to the compression acting surface side of the inner peripheral side sheet portion.

7. The sphygmomanometer cuff according to claim 1, wherein the cushion material is a sheet-like member of even thickness.

8. The sphygmomanometer cuff according to claim 1, wherein the cushion material is a member in which thickness is changed in at least one of either axial direction or peripheral direction of the sphygmomanometer cuff.

9-10. (canceled)

11. A sphygmomanometer comprising:
the sphygmomanometer cuff according to claim 1;
an inflation/contraction mechanism for inflating/contracting the fluid bag;
a pressure detection unit for detecting pressure in the fluid bag; and
a blood pressure value calculation unit for calculating a blood pressure value based on pressure information detected by the pressure detection unit.
